(19) United States
(12) Patent Application Publication
(10) Pub. No.: US 2020/0311922 A1
(43) Pub. Date: Oct. 1, 2020

(54) CELL OBSERVATION SYSTEM AND INFERENCE MODEL GENERATING METHOD

(71) Applicant: OLYMPUS CORPORATION, Tokyo (JP)

(72) Inventors: Satoshi HARA, Tokyo (JP); Jun SUGAWARA, Tokyo (JP); Keiji OKADA, Tokyo (JP); Mikako HORI, Tokyo (JP); Takuma DEZAWA, Tokyo (JP); Masaaki YAMAGISHI, Tokyo (JP); Kazuhiko OSA, Tokyo (JP); Osamu NONAKA, Sagamihara-shi (JP)

(73) Assignee: OLYMPUS CORPORATION, Tokyo (JP)

(21) Appl. No.: 16/829,162

(22) Filed: Mar. 25, 2020

(30) Foreign Application Priority Data
Mar. 27, 2019 (JP) 2019-060850

(51) Int. Cl. G06T 7/00; C12M 1/34; G06T 1/20

(52) U.S. Cl. CPC G06T 7/0012; G06T 2207/30024; G06T 1/20; C12M 41/36

(57) ABSTRACT

An cell observation system, comprising an image sensor capable of movement in a horizontal direction, and a processor, wherein the processor infers position where a colony will be generated or grown from position information or shape information of a plurality of cells within an image, based on the image of the cells that has been acquired by the image sensor, and controls position of the image sensor so as to perform imaging at the inferred position.

### CROSS-REFERENCE TO RELATED APPLICATIONS

Benefit is claimed, under 35 U.S.C. § 119, to the filing date of prior Japanese Patent Application No. 2019-060850 filed on Mar. 27, 2019. This application is expressly incorporated herein by reference. The scope of the present invention is not limited to any requirements of the specific embodiments described in the application.

### BACKGROUND OF THE INVENTION

### 1. Field of the Invention

The present invention relates to a cell observation system that observes cells that are cultivated in a culture medium that has been disposed in a stabilized environment, such as within an incubator, and to a generation method for an inference model for observing a cell.

### 2. Description of the Related Art

Various observation devices have been proposed in which cells are cultivated within a vessel that has been filled with a culture medium, and these cells are observed. For example, WO2009/031283 (Hereafter referred to as “patent publication 1”) proposes a cultivation device in which microscope images are formed in order at observation points and these taken images are stored, and it is possible to retrieve only images that satisfy specified conditions, from among the stored images. Also, in WO2010/098105 (hereafter referred to as “patent publication 2”), there is proposed a cultivation state evaluation device in which microscope images are formed in order at observation points, and evaluation information for evaluating cultivation state of cells is generated from those taken images. In Japanese patent laid-open No. 2010-504086 (hereafter referred to as “patent publication 3”) a device is proposed in which cells and cell colonies having particular characteristics are selected, and cells and cell colonies that have been selected are removed.

Field of vision when forming images of cells within a culture vessel with a microscope etc. is narrow because generally the purpose is magnified observation, and forming images of cells etc. within a culture vessel using a microscope takes time. For example, if cells are cultivated in order to acquire multifunctional stem cells such as iPS cells, or in order to create a monoclonal cell population having desired characteristics, colonies are increasingly formed. However, not all cells that are cultivated constitute a specific expected colony, and so if images are formed at all points within the culture vessel efficiency will be bad and time taken to form images will become long. With patent publications 1 to 3 described above, images are formed of cells within a culture vessel at all points that have been set, and efficiency is not good. Also, in patent publications 2 and 3, although evaluation of cells is described there is no description whatsoever regarding selection of positions for imaging and observation based on results of cell evaluation.

### SUMMARY OF THE INVENTION

The present invention provides a cell observation system that is capable of changing imaging and observation points in accordance with change in cultivation state (generation state of a colony), when forming images of and observing cells etc., and a generating method for an inference model for observing a cell.

A cell observation system of a first aspect of the present invention comprises an image sensor capable of movement in a horizontal direction, and a processor, wherein the processor infers position where a colony will be generated or grown from position information or shape information of a plurality of cells within an image, based on the image of the cells that has been acquired by the image sensor, and controls position of the image sensor so as to perform imaging at the inferred position.

An inference model generating method of a second aspect of the present invention comprises, acquiring image data that has been formed in time series of appearance of cell cultivation that has reached cell colony formation, designating image portions where there will be colonization, among the image data that has been acquired, as annotation, making image data that has been designated with this annotation into training data, and generating a colonization inference model that has input of cell images before colonization, and output of expected colonization positions, using the training data.

A non-transitory computer-readable medium of a third aspect of the present invention, storing a processor executable code, which when executed by at least one processor, performs a cell observation method, the cell observation method comprising inferring position where a colony will be generated or grown from position information or shape information of a plurality of cells within an image, based on the image of the cells that has been acquired by an image sensor that is capable of movement in a horizontal direction, and controlling position of the image sensor so as to perform imaging at the inferred position.

### BRIEF DESCRIPTION OF THE DRAWINGS

FIG. 1A to FIG. 1E are drawings showing appearance of a colony occurring from cells, and is one example of observation using the cell observation device of one embodiment of the present invention.

FIG. 2 is an external drawing of a cell observation system of one embodiment of the present invention comprising a cell observation device, and an information terminal device.

FIG. 3 is a block diagram mainly showing the electrical structure of a cell observation system of one embodiment of the present invention comprising a cell observation device and an inference device.

FIG. 4A and FIG. 4B are examples of training images used when generating an inference model, in the cell observation system of one embodiment of the present invention.

FIG. 5 is a flowchart showing operation of an imaging section of the cell observation system of one embodiment of the present invention.

FIG. 6A and FIG. 6B are flowcharts showing operation of an information terminal device of the cell observation system of one embodiment of the present invention.

FIG. 7A and FIG. 7B are drawings showing icon display, in an information terminal device of the cell observation system of one embodiment of the present invention.

FIG. 8A and FIG. 8B are external drawings showing a first modified example of display images of an information terminal device, in the cell observation system of one embodiment of the present invention comprising a cell observation device and an information terminal device.

FIG. 9A to FIG. 9C are external drawings showing a second modified example of display images of an information terminal device, in the cell observation system of one embodiment of the present invention comprising a cell observation device and an information terminal device.

FIG. 10A to FIG. 10C are drawings showing display of combined images, in an information terminal device of the cell observation system of one embodiment of the present invention.

FIG. 11A and FIG. 11B are drawings showing display of colony position and taken images, in an information terminal device of the cell observation system of one embodiment of the present invention.

FIG. 12 is a flowchart showing another example colonization determination, in the cell observation system of one embodiment of the present invention.

### DETAILED DESCRIPTION OF THE PREFERRED EMBODIMENTS

In the following, description will be given of one embodiment of the present invention, having been applied to a cell observation system comprising a cell observation device and an information terminal device.

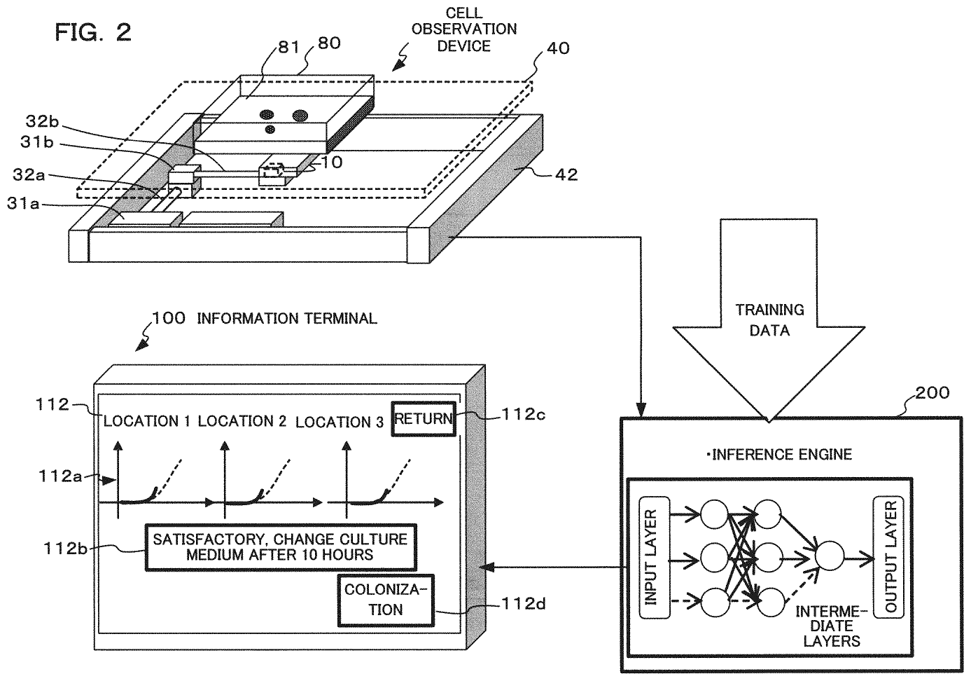

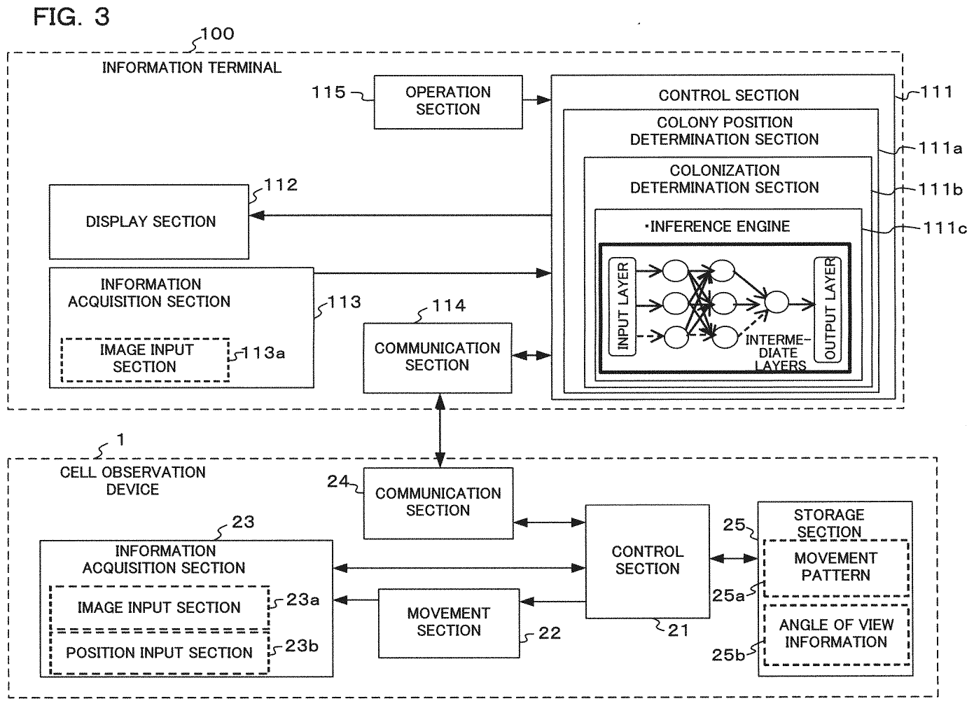

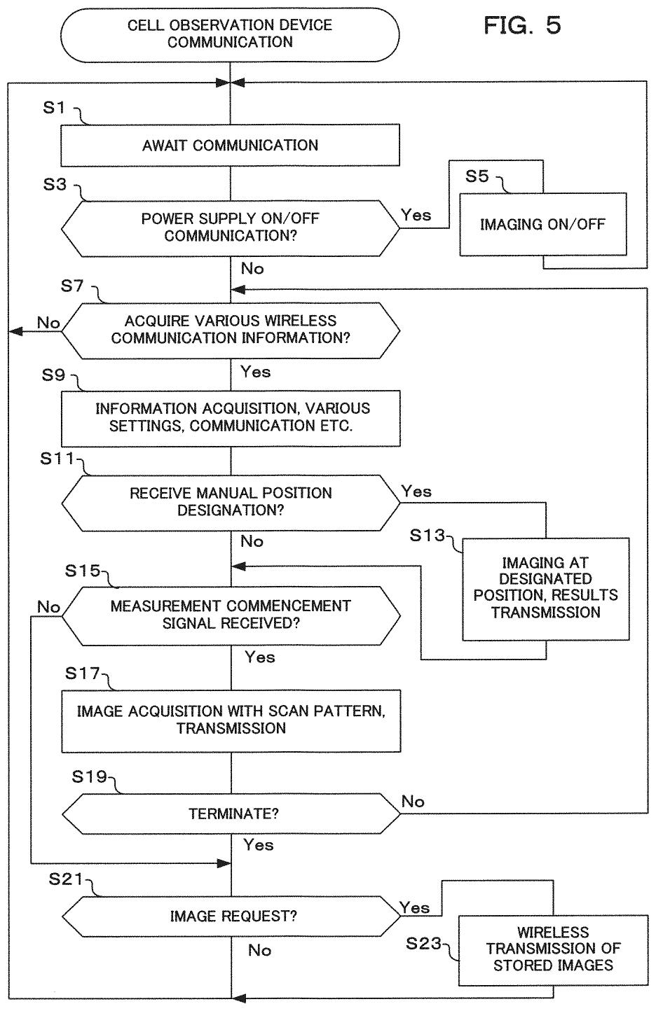

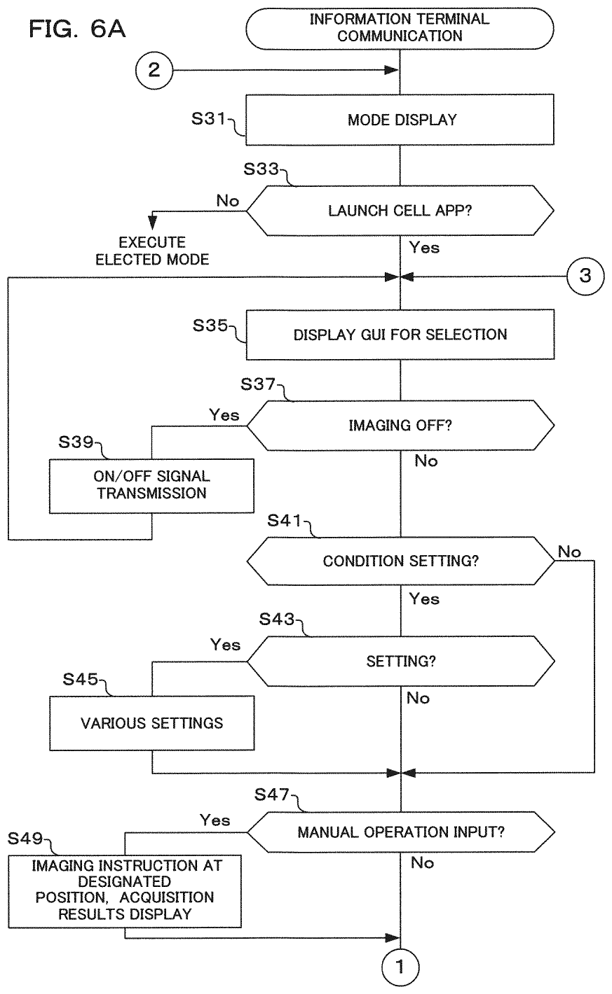

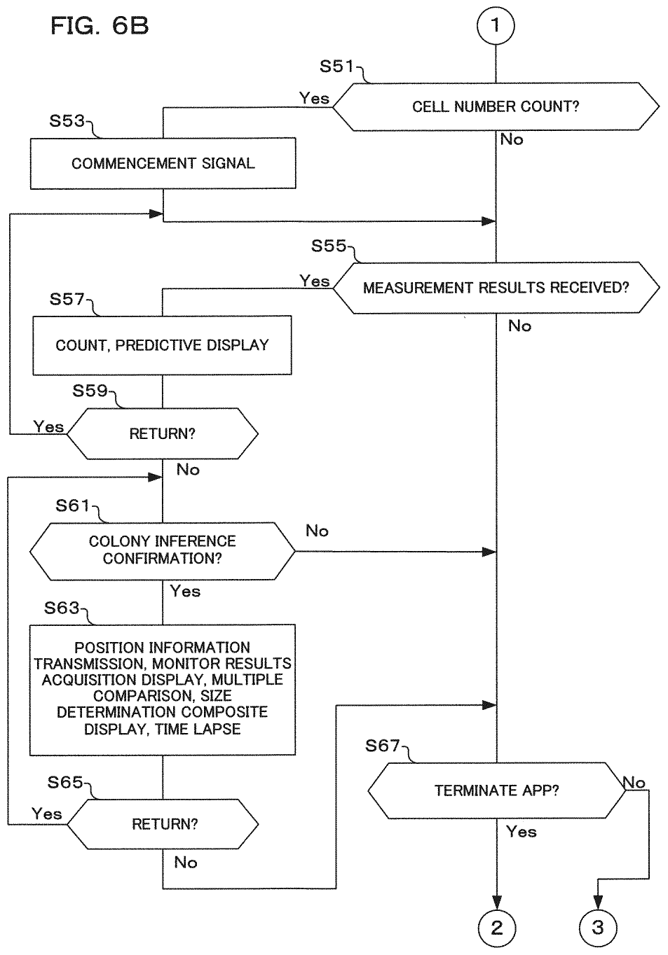

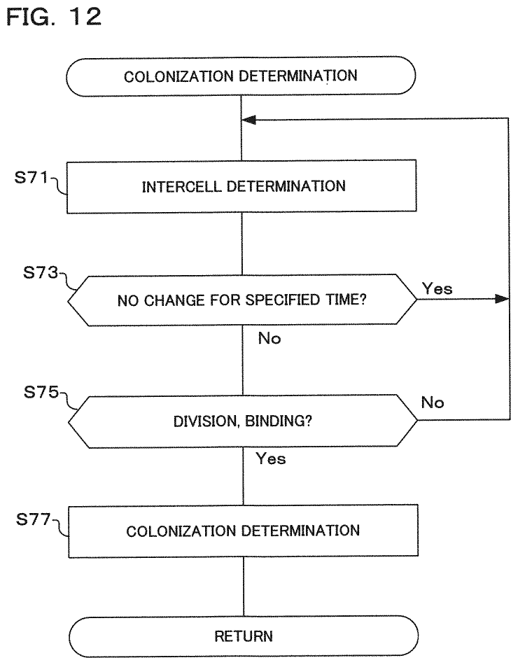

In the cell observation system of this embodiment, cell images are acquired, and positions where colonies of cells are being formed, and positions where formation is expected, are inferred based on the cell images (refer to the inference engine 111c in FIG. 3, and to S61 and S63 in FIG. 6B). Positions where cells are imaged, by an imaging section (camera section 10 in FIG. 2, image input section 23a in FIG. 3) are adjusted based on these positions of colonies and formation expected positions that have been inferred (refer to S17 in FIG. 5). It should be noted that cell colony formation positions and formation expected positions may be obtained using logic using a flowchart, and is not limited to inference (FIG. 12).

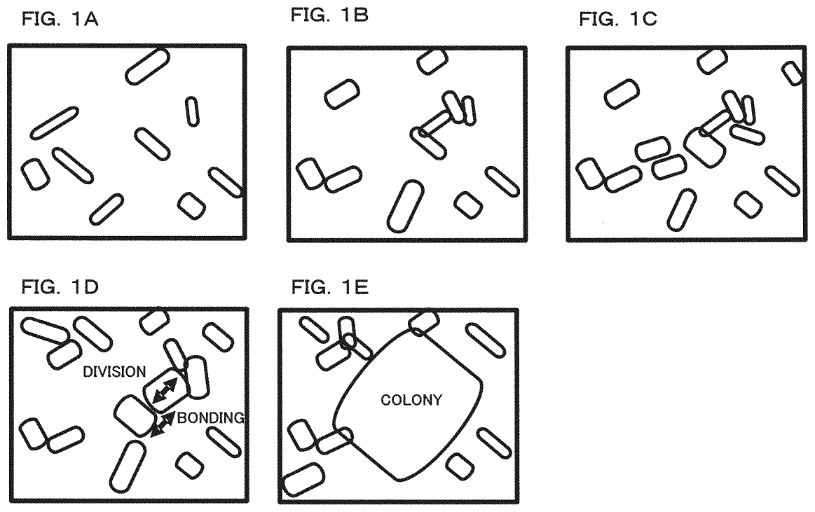

As was described previously, with the cell observation system of this embodiment, at the time of cell cultivation and preparing of colonies, position where colonies have been formed and positions where formation is expected are predicted, and shooting is performed using the imaging section based on these predicted positions etc. Therefore, initially, description will be given of how colonies are formed at the time of cell cultivation, using FIG. 1A to FIG. 1E.

Research into multifunctional stem cells having the ability to be differentiated into various structures has been gathering attention in the field of regenerative medicine. As multifunctional stem cells, “embryonic stem cells (ES cells)” are known, and recently “induced pluripotent stem cells (iPS cells)” etc. that have been created artificially by introducing genes into somatic cells are also known. Human multifunctional stem cells that it is assumed would be used in a patient's treatment are cultivated as colonies. A colony is a cell aggregation of cells resulting from cells dividing and adhering within a culture medium. A plurality of cultured cells induced from a single progenitor cell by cell replication is called monoclonal. It is also possible to apply this embodiment to this type of monoclonal cell cultivation. Processes etc. to plant and inherit cells called medium exchange and subculture are performed, and a colony is managed as a moderate small mass. If this management is neglected, “differentiation,” to change into cells of a type where cells that are not specialized are made specialized, occurs.

Accordingly, for example, although technology is necessary to manage this undifferentiated cell colony for appropriate culturing, cells died out due to detailed conditions of cultivation, and colonies arose that were different to those expected. Since cultivation takes a number of days, efficiency was bad with determination after cells dying, colonies other than those expected being possible etc.

If transcription factors are put into human skin cell derived fibroblasts, initialization of a nucleus for which gene expression state has changed within cells due to this gene transfer occurs, and creation of iPS commences from within a few hours to less than 48 hours after introduction of factors. There is change into a shape of an iPS cell colony while performing fission in a similar shape to the original fibroblasts. However, there may be cases where colony formation fails due to repeated self-replication semi-permanently with differentiation potency maintained. This state is therefore dealt with by detecting at an early stage.

FIG. 1A to FIG. 1E are schematic drawings showing change in cells being cultivated. FIG. 1A is initial appearance, and FIG. 1B, FIG. 1C . . . FIG. 1E show appearance with the cells changing over time. If genes are introduced into the cells, then after cultivation, at about one week, colonies form as shown in FIG. 1E. As shown in FIG. 1D, until a day before colony formation, prediction from the state of FIG. 1D to FIG. 1E is difficult, due to the shape of the original fibroblast. This is because division and binding (colonization) advance at substantially the same time as cell division and cohesion as a result of interaction between cells that have contacted (adhered). With this embodiment, colonization is predicted based on state change of both cells at the time a plurality of cells come into contact.

Next, the structure of the cell observation system of this embodiment will be described using FIG. 2. The cell observation system comprises a cell observation device 1, information terminal 100, and inference engine 200. The cell observation device 1 and a cell culture vessel 80 are arranged within an incubator, and the information terminal 100 and the inference engine 200 are arranged outside the incubator.

The cell culture vessel 80 is mounted on a transparent top board 40 of the cell observation device 1, images of a specimen 81 that has been cultivated in the cell culture vessel 80 are formed through the transparent top board 40 and taken image data can be acquired. This means that it is possible to cultivate cells inside an incubator etc., with environment maintained, and to perform measurement and observation of a specimen 81 or the like in the information terminal 100 etc. outside of the incubator. Since observation and measurement of the cells that have been cultivated inside the incubator are performed remotely, it is desirable for the cell observation device 1 to have an energy saving and high manufacturing reliability design.

There is an imaging section (image input section 23a in FIG. 2) comprising a photographing lens, image sensor, and imaging control circuit in the camera section 10, and the imaging section forms images of the specimen 81 and generates image data. A light source such as an LED (Light Emitting Diode) for illumination is arranged in the imaging section. Illumination light of the LED etc. is irradiated in the direction of the top board 40 and the cell culture vessel 80, reflected by the cover of the cell culture vessel 80, and the specimen 81 is illuminated by this reflected light that has passed through the specimen. It should be noted that the light source such as the LED etc. may be arranged above the cell culture vessel 80, and the specimen 81 may be illuminated by light that passes through. The illumination light source may also use a light source other than an LED.

Also, a wireless communication device (refer to communication section 24 in FIG. 3) is arranged inside the cell observation device 1, and is capable of wireless communication with a communication section 114 within the information terminal 100 that is arranged externally to the cell observation device 1. Detailed electrical structure of the camera section 10 within the cell observation device 1 will be described later using FIG. 3.

The camera section 10 is capable of movement in the X axis direction and the Y axis direction, that is, can be moved within a plane in the horizontal direction. Specifically, the camera 10 is held on an X feed screw 32b, and is capable of movement in the X axis direction by rotation of the X feed screw 32b. The X feed screw 32b is driven to rotate by the X actuator 31b. The X actuator 31b is held on the Y feed screw 32a, and is capable of movement in the Y axis direction by rotation of the Y feed screw 32a. The Y feed screw 32a is driven to rotate by the Y actuator 31a. The control section 21 (refer to FIG. 3) performs drive control for the Y actuator 31a and the X actuator 31b, and performs drive control of the camera section 10 in the X axis and Y axis directions in accordance with a procedure that has been preprogrammed. It is also possible for the user to move the camera section 10 to a specified position, and in this case, since a manual operation is instructed by the information terminal 100, the movement control section 33 moves the camera section 10 in accordance with the user's instruction (refer to S11 and S13 in FIG. 5, and to S47 and S49 in FIG. 6A).

It should be noted that a built-in power supply battery is provided inside the cell observation device 1, and supplies power to the Y actuator 31a, X actuator 31b, and camera section 10, and a communication line is also provided for bidirectional communication of control signals between each of the sections. With this embodiment it is assumed that a power supply battery is used as the power supply, in order to simplify arrangement of the cell observation device 1 within the incubator, but this is not limiting, and supply of power may also be implemented using an AC power supply. It is also assumed that control signals between each of the sections are interchanged by means of wired communication, but it is also possible to use wireless communication.

The above described camera section 10, Y actuator 31a, X actuator 31b, Y feed screw 32a, and X feed screw 32b, are arranged inside a housing that is made up of the top board 40 and an outer housing 42. The top board 40 and outer housing 42 constitute an encapsulating structure such that moisture does not infiltrate into the inside from outside. As a result, the inside of the housing constituted by the top board 40 and the outer housing 42 are not subjected to high humidity, even if the inside of the incubator is high humidity. On the other hand, the cell culture vessel 80 that has been placed on the top board 40 of the cell observation device 1 is maintained at a temperature and humidity that have been adjusted by the incubator.

It is possible to mount the cell culture vessel 80 on the upper side of the transparent top board 40, and it is possible to fill a culture medium into the inside of the cell culture vessel 80 and cultivate a specimen 81 (cells). The lens of the camera section 10 forms images of the culture medium inside the cell culture vessel 80 through the transparent top board 40, and it is possible to observe images of cells etc. At the time of this imaging a light source such the LED illuminates the specimen 81, as was described previously. Since images of the cells within the cell culture vessel 80 are formed by the camera section 10, the bottom surface of the cell culture vessel 80 (side in contact with the top board 40) is preferable transparent.

The information terminal 100 is external to the incubator, and performs control of the cell observation device 1 from outside. Specifically, the information terminal 100 has the communication section 114, as shown in FIG. 3, and is capable of wireless communication with the communication section 24 within the cell observation device 1. This means that it is possible for the information terminal 100 to perform communication from a position that is isolated from the cell observation device 1, and it is possible to move the camera section 10 and to receive image data that has been acquired by the camera section 10. It should be noted that the information terminal 100 may be a dedicated unit, and an information terminal device such as a smartphone may also double as the operation section. Further, an operation section that belongs to a computer such as a personal computer (PC) or a server may also be used for the information terminal 100.

The information terminal 100 also has a display section 112, and it is possible to display images that have been acquired by the cell observation device 1 on this display section 112. Also, the display section 112 shows a cell number graph 112a for each of position (location) 1, position 2, position 3, . . . , within the cell culture vessel 80, as shown in FIG. 2, in a case where count mode (with predictive display) has been set. This graph 112a shows count result for every position (solid line) and predicted number of cells (dashed line). Cultivation information 112b relating to the cell culture is also shown. With the example shown in FIG. 2, as culture information there is that cell culture is “good”, and the fact that it is necessary to “replace culture medium” “after 10 hours” is displayed. A “return” icon 112c is an icon for returning the screen of the display section 112 to a previous screen. A “colonization” icon 112d shows that a location being shown is colonized. With the example shown in FIG. 2, an icon is only shown underneath location 3, and so it shows that only location 3 has been colonized, while locations 1 and 2 are not colonized.

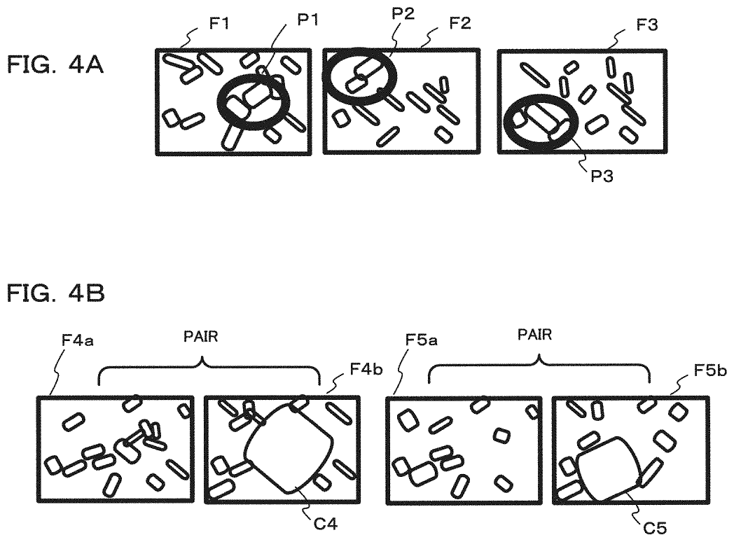

The inference engine 200 generates an inference model for inferring colonization. The inference engine 200 may also be provided within the information terminal 100, but is provided in a server that is capable of being connected to through the Internet etc. The inference engine 200 is input with training data, and generates an inference model for performing inference to predict colonization. This training data will be described later using FIG. 4A and FIG. 4B. An inference model that has been generated by the inference engine 200 is held in the inference engine 111c within the information terminal 100, and is used in prediction of the occurrence of colonies.

Next, the electrical structure of the cell observation device 1 and the information terminal 100 of this embodiment will be described using FIG. 3. The cell observation device 1 comprises a control section 21, movement section 22, information acquisition section 23, communication section 24 and storage section 25. The information acquisition section 23 has an image input section 23a and a position input section 23b, and acquires various information. Further, an operation section for operational checks etc. at a device unit may also be installed, and a display section that display results of operation checks may also be installed.

The image input section 23a is arranged within the camera section 10 shown in FIG. 2, and comprises a photographing lens, image sensor, imaging control circuit etc. The image input section 23a converts images of the specimen 81 to image data, and outputs the image data to the control section 21. As was described previously, the camera section 10 is capable of moving in a horizontal direction, and it is possible to acquire images of the specimen 81 at a position that has been designated. The camera section 10 is placed underneath the specimen 81 (cells) within the vessel 80, and can form images of the specimen 81. The image input section 23a is capable of movement in the horizontal direction, and functions as an imaging section that forms images of cells cultivated within the vessel. An image sensor within the image input section 23a functions as an image sensor that is capable of movement in the horizontal direction.

The position input section 23b is input with position information of the camera section 10, that is, shooting position. Regarding the shooting position, position sensors such as an X axis direction encoder and a Y axis direction encoder for measuring position of the camera section 10 may be provided, and output of this position sensor input as the shooting position. Also, a position control signal when moving the camera section 10 with the movement section 22 may be input and detected. It should be noted that the camera section 10 may have an auxiliary light source necessary for observation installed, and may also use a separate light source.

The movement section 22 comprises drive sources (for example, motors or the like) such as the previously described X axis actuator 31b and the Y axis actuator 31a, and a control circuit that controls drive of these drive sources. The movement section 22 moves the camera section 10 based on control signals from the control section 21. The movement section 22 functions as a movement section that controls position such that images are formed by the imaging section at central positions of colonies, based on colony positions that have been determined by a colony position determination section. The actuators 31a and 31 within the movement section 22 function as actuators that move the image sensor in the horizontal direction.

The communication section 24 has a communication circuit, and performs communication with the communication section 114 within the information terminal 100. The cell observation device 1 transmits images of cells that have been acquired by the image input section 23a, and position information at the time of shooting, to the information terminal 100 by means of the communication section 24. Also, the information terminal 100 analyzes images that have been received from the cell observation device 1, and transmits position information where it is predicted that colonies of cells will be generated to the cell observation device 1 by means of the communication section 24. Control of shooting position is performed based on colony generation prediction positions that have been received from the control section 21.

The storage section 25 has an electrically rewritable non-volatile memory and an electrically rewritable volatile memory, and stores movement patterns 25a and angle of view information 25b. Memory is arbitrary storage medium such as RAM (Random Access Memory), for example. Non-volatile memory is, for example, a hard disk, Flash memory etc. The movement patterns 25a are patterns in which the camera 10 is moved by the movement section 22. These movement patterns are received in advance from the information terminal 100 and stored. The control section 21 reads out movement pattern 25a, performs control of the movement section 22, and acquires images using the image input section 23a. Also, the information terminal 100 predicts locations where colonies will occur based on images that have been acquired during cell cultivation, and if a movement pattern has been generated based on prediction results the cell observation device 1 receives a movement pattern based on this prediction result and stores as a movement pattern 25a. Specifically, the recording section 25 functions as a memory that is capable of storing inference position that have been inferred by a processor.

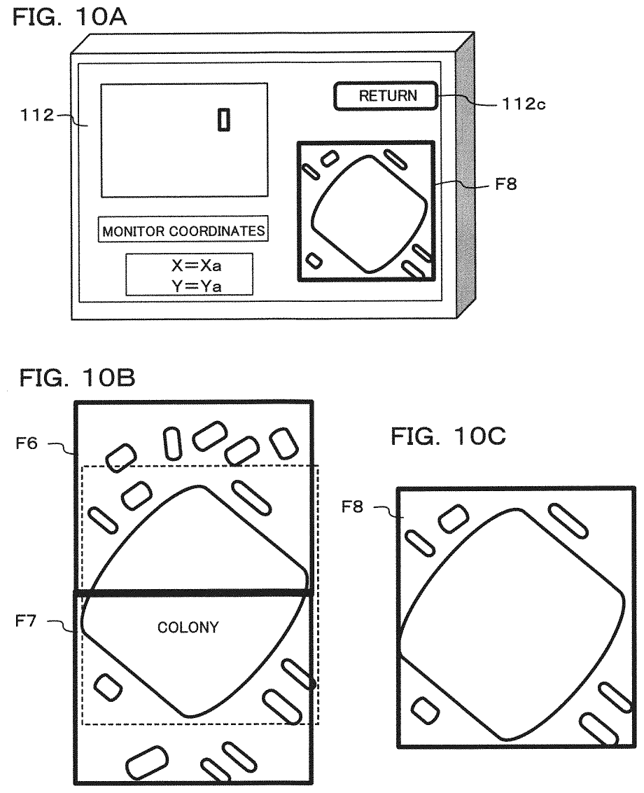

The angle of view information 25b is focal length information of a photographing lens of the image input section 23a. As a method of acquiring and displaying an entire colony, a plurality of images that have been acquired by the image input section 23a (imaging unit) may be combined, and the entire colony displayed (refer, for example, to FIG. 10B).

The control section 21 is a processor having a CPU (Central Processing Unit), memory that stores programs, and peripheral circuits, and performs overall control of the cell observation device 1 in accordance with programs. As control performed by the control section 21, for example, the camera 10 is moved by the movement section 22 in accordance with a movement pattern 25a that has been stored in advance or a movement pattern 25a that has been instructed from the information terminal 100, and images are acquired by the image input section 23a at positions that have been designated. Images that have been acquired are transmitted to the information terminal 100 by means of the communication section 24.

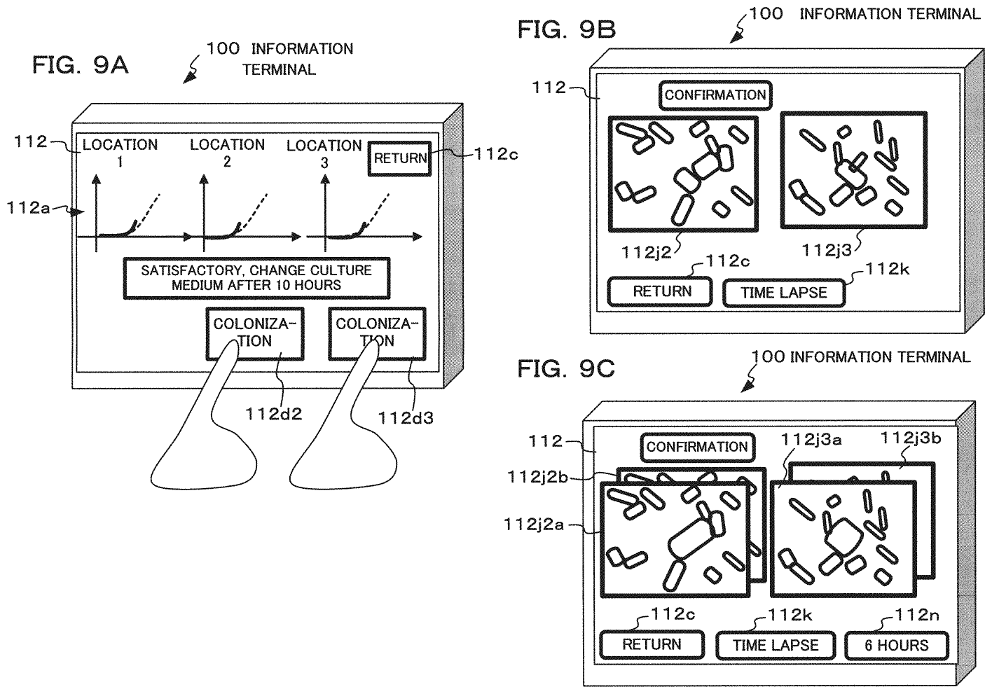

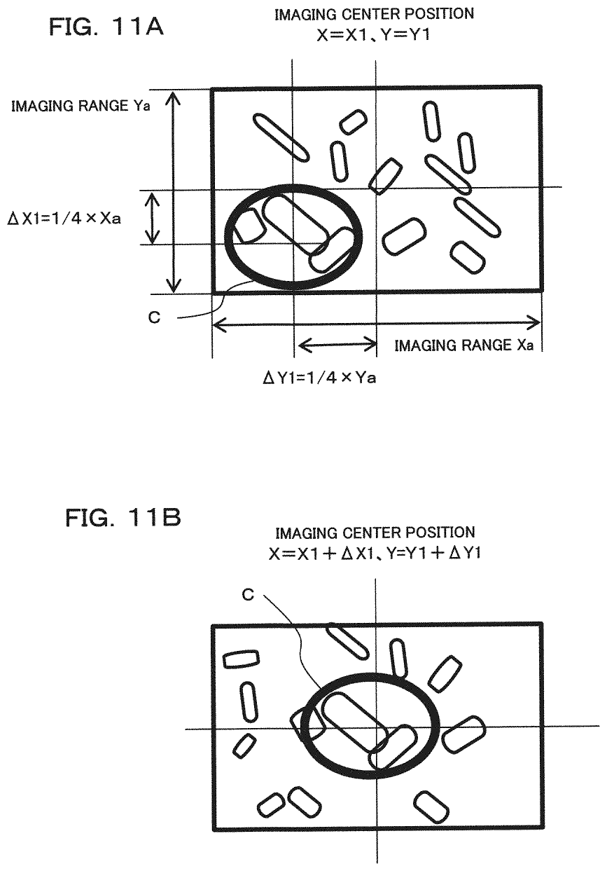

Also, in a case where a position where there will be generation or growth of colonies has been inferred by the information terminal 100, since that position is transmitted (refer to S63 in FIG. 6B) the camera 10 is moved by the movement section 22 based on that position (refer to S17 in FIG. 5). Specifically, the above described processor controls position of the image sensor so as to perform imaging at the inferred position. Also, the processor controls actuators based on the inferred position so that the image sensor take images at center positions of colonies (refer, for example, to S17 in FIG. 5, and to FIG. 11B). The processor controls the image sensor so as to take time-lapse images, at specified time intervals, of cells or colonies at the inferred positions (refer, for example, to S17 in FIG. 5, S63 in FIG. 6B, and to FIG. 9B and FIG. 9C).

The control section 21 functions as a time-lapse control section that performs imaging control of colonies by the imaging section at specified time intervals, if an instruction for time lapse has been received from the information terminal 100. This time lapse control section takes pictures of colonies at colony positions that have been predicted, at specified time intervals.

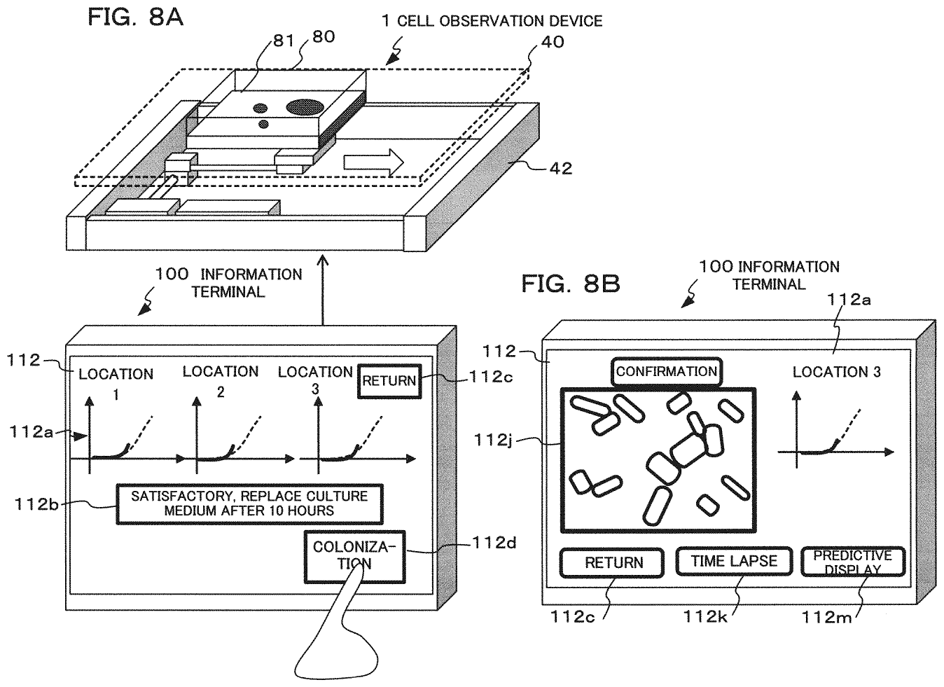

The information terminal 100 comprises a control section 111, display section 112, information acquisition section 113 and communication section 114. The display section 112 has a monitor screen for display, and displays images of cells that have been received from the cell observation device 1. Also, as shown in FIG. 2, results of counting cells are subjected to graph display, and besides this, information relating to cell cultivation is also displayed. It should be noted that a modified example of the display section 112 will be described later using FIG. 8A to FIG. 8B, and FIG. 9A to FIG. 9C. The display section 112 functions as a display that is capable of display images of cells or colonies that have been acquired by the image sensor. The display section 112 functions as a display section that displays images of colonies that has been acquired by the imaging section. This display section is capable of comparing and displaying a plurality of colonies (refer to FIG. 9A to FIG. 9C). Also, the display section performs adjustment so as to include an entire colony, in a case where a colony is spread over a plurality of images of cells (refer to FIG. 10A to FIG. 10C). The information acquisition section 113 has an image input section 113a. The image input section 113a comprises a photographing lens, image sensor, and imaging control circuit, etc., and acquired images.

The communication section 114 has a communication circuit, and performs communication with the communication section 24 within the cell observation device 1. As was described previously, by means of this communication section 114 images that have been acquired by the cell observation device 1 are received, and information such as of colony generation positions that have been inferred by the inference engine 111c are transmitted to the cell observation device 1. An operation section 115 is an interface for the user to input instructions to the cell observation device 1 and the information terminal. As the operation section 115, there are operation members such as switches for operation, and a touch panel that is capable of touch operations.

The control section 111 is a processor having a CPU (Central Processing Unit), a memory that stores programs, and peripheral circuits, and performs overall control of the information terminal 100 in accordance with programs. As control of the information terminal 100, for example, future positions where colonies will occur are predicted based on images of cells that have been received from the cell observation device 1 (refer, for example, to FIG. 1A to FIG. 1E), and instructions are issued for the cell observation device 1 so as to take images of cells, based on the predicted positions.

Specifically, the above described processor infers positions where colonies will be generated or grow from position information or shape information of a plurality of cells within an image based on images of cells that have been acquired by the image sensor (refer, for example, to S61 in FIG. 6B), and controls position of the image sensor so as to perform imaging at inferred positions (refer, for example, to S63 in FIG. 6B). The inferred positions mentioned above are positions where it is predicted that colonies will be generated (refer, for example, to S61 in FIG. 6B). Also, the processor controls actuators based on the inferred position so that the image sensor takes images at center positions of colonies (refer, for example, to S17 in FIG. 5, to S63 in FIG. 6B, and to FIG. 11B). The processor controls the image sensor so as to take time-lapse images at specified time intervals of cells or colonies at the inferred positions (refer, for example, to S17 in FIG. 5, S63 in FIG. 6B, and to FIG. 9B and FIG. 9C). The processor analyzes a plurality of images that have been acquired by the image sensor at the inferred positions, measures a number of cells within the images, and outputs a change in number of cells overtime to the display (refer, for example, to S63 in FIG. 6B, and to FIG. 8A, FIG. 8B and FIG. 9A etc.).

The control section 111 comprises a colony position determination section 111a, a colonization determination section 111b, and an inference engine 111c. The inference engine 111c holds an inference model that has been received from the inference engine 200, and performs inference. This inference engine 111c has image that have been received from the cell observation device 1 input to an input layer, performs determination as to whether or not a colony is occurring using the inference model, determines positions where colonies are occurring, and outputs determination results (inference results) from an output layer. These determinations are not limited to the current time, and prediction of future occurrences may also be performed.

The colonization determination section 111b and colony position determination section 111a within the control section 111 predict positions where colonies will occur in the future, or where colonies have grown, based on inference results from the inference engine 111c, and transmit positions where images should be acquired in the cell observation device 1 to the cell observation device 1 based on the prediction results. Also, the control section 111 has an image analysis section (image analysis circuit), as a peripheral circuit, that identifies cells and counts a number of cells based on images that have been acquired by the cell observation device 1. It should be noted that identification of cells may be identification using the inference engine 111c and counting of the number of cells.

An inference model used in the inference engine 111c is generated in the information terminal 100, or in an inference model generating device that has been provided within a server that is provided externally to the information terminal 100. Generation of this inference model involves first designating image portions where there is colonization, within image data that has been acquired by imaging of appearance of cell cultivation leading to cell colony formation, in time series, as annotation, and making image data in which this annotation has been designated into training data. Next, a colonization inference model is generated using the training data, with input made cell images before colonization, and output made expected site of colonization. The inference engine 111c functions as an inference engine having a colonization inference model having cell images made into training data. The above described inference model outputs information on inferred positions where colonies will be generated, based on input of images that were acquired by the image sensor (refer, for example, to S63 in FIG. 6B). Also, the inference model outputs determinations results as to whether cultivation is good or bad based on images or information on inferred position.

The colony position determination section 111a determines positions where colonization will occur, based on images that have been received from the cell observation device 1. Also, the colonization determination section 111b determines whether or not colonies have occurred. The colonization determination section 111b functions as a determination section that changes position by moving the imaging section in the horizontal direction and determines a colony based on images of cells that have been acquired by the imaging section. The colony position determination section 111a functions as a colony position determination section that determines positions of colonies based on a movement position and imaging range of an imaging section when a colony has been determined. The determination section described above determines colony position by predicting that a colony will arise from position and shape information of a plurality of cells.

Next, training data for performing deep learning in the inference engine 200 and generating an inference model will be described using FIG. 4A and FIG. 4B. FIG. 4A shows an example where annotation has been applied to positions P1 to P3 where colonies occur, in cell images F1 to F3. Annotation may be applied to positions P1 to P3 where a colony has actually occurred, but annotation may also be applied to positions P1 to P3 on images before a colony is embodied. In this case, at positions where a colony has actually occurred, annotation may be applied to corresponding positions in image before a colony occurred. Also, an experienced specialist may apply annotation to positions where it is predicted that a colony will occur.

FIG. 4B is an example where an image where a colony has actually occurred is paired with an image before a colony occurred that was taken at that position, and annotation is applied using this pair of images. Cell images F4a and F4b are a pair of images, and cell images F5a and F5b are also a pair of images. Since colonies C4 and C5 are occurring in cell images F4b and F5b, annotation may be performed at corresponding positions in cell images F4a and F5a. In this way, by annotating a location where occurrence is expected in an image before a colony has actually occurred, training data for inference model generation is created. If deep learning is performed using this training data it is possible to generate an inference model that is capable of prediction locations where colonies will occur.

It should be noted that training data may also be generated for inference of locations where it is predicted that colonies will not occur. In this case, annotation is applied at positions in cell images F1 to F3, F4a and F5a where it is predicted that colonies will not occur, to create negative training data. By performing deep learning using this training data it becomes possible to infer locations where colonies will not occur.

In this way, with this embodiment, by counting images and cells in which progress of cell cultivation is observed, etc., characteristics of change are inferred, such as being able to predict future situations using data having a proven track record, such as colonization that has been acquired in advance. In order to do this annotation is performed to append good or bad determination and position information to data before colonization. Specifically, whether or not colonies will be formed is inferred based on time series images of cells that have been acquired by an imaging section that takes sequential images in time series of cells that have been cultivated in a vessel. When creating an inference model that will perform this inference, position and shape information of a plurality of cells is used. In other words, effective practical use is made of image data that was obtained by imaging a cell culture leading to generation of colonies of cells in time series. Specifically, among image data that was obtained by imaging appearance of a cell culture leading to generation of colonies of cells in time series, data that was obtained by designating image portions where there is colonization as annotation is made training data, and a colonization inference model that has input of cell images before colonization and output of portions where colonization is expected is generated.

With this embodiment, although whether or not cell cultivation has proceeded as expected is determined by generation of colonies, obviously whether or not cell cultivation has proceeded as expected may also be determined using another method. In other words, if there are time series imaging results and cell count data for the same culture positions that have resulted from a cell cultivation that was cultivated as expected, then since training data for creating an inference model can be obtained from characteristics of that change, prediction determination as to whether or not cultivation will proceed as expected may be performed using this inference model.

Also, if there is a purpose for assuming efficiency preferred, such as of ascertaining whether cultivation is good or bad at an early stage, a case where progress is not as expected may be determined, and in this case also, a similar approach can be applied. That is, if there are time series imaging results and cell count data for the same culture positions that have resulted from cell cultivation that was not cultivated as expected, training data for creating an inference model for inferring that cultivation will not be performed well is obtained from characteristics of that change. An inference model is generated using this training data, and prediction determination of whether cultivation will progress as expected may be performed using this inference model. Among image data that has been acquired by imaging appearance of cells that have been cultivated leading to cell cultivation success, in time series, data that has been acquired by designating portions of cells that have been cultivated as expected, or not as expected, as annotation within an image are made training data, and an inference model for cell cultivation success or failure display, with inputs of cell images and outputs of image portions where there are cells that have been cultivated as expected, or not as expected, may be generated.

Here, deep learning will be described. “Deep Learning” involves making processes of “machine learning” using a neural network into a multilayer structure. This can be exemplified by a “feedforward neural network” that performs determination by feeding information forward. The simplest example of a feedforward neural network should have three layers, namely an input layer constituted by neurons numbering N1, an intermediate layer constituted by neurons numbering N2 provided as a parameter, and an output layer constituted by neurons numbering N3 corresponding to a number of classes to be determined. Each of the neurons of the input layer and intermediate layer, and of the intermediate layer and the output layer, are respectively connected with a connection weight, and the intermediate layer and the output layer can easily form a logic gate by having a bias value added.

While a neural network may have three layers if simple determination is performed, by increasing the number of intermediate layers it becomes possible to also learn ways of combining a plurality of feature weights in processes of machine learning. In recent years, neural networks of from 9 layers to 15 layers have become practical from the perspective of time taken for learning, determination accuracy, and energy consumption. Also, processing called “convolution” is performed to reduce image feature amount, and it is possible to utilize a “convolution type neural network” that operates with minimal processing and has strong pattern recognition. It is also possible to utilize a “recursive neural network” (fully connected recurrent neural network) that handles more complicated information, and with which information flows bidirectionally in response to information analysis that changes implication depending on order and sequence.

In order to realize these techniques, it is possible to use conventional general purpose computational processing circuits, such as a CPU or FPGA (Field Programmable Gate Array). However, this is not limiting, and since a lot of processing of a neural network is matrix multiplication, it is also possible to use a processor called a GPU (Graphic Processing Unit) or a Tensor Processing Unit (TPU) that are specific to matrix calculations. In recent years a “neural network processing unit (NPU) for this type of artificial intelligence (AI) dedicated hardware has been designed to be capable being integratedly incorporated together with other circuits such as a CPU, and there are also cases where they constitute some parts of processing circuits.

Besides this, as methods for machine learning there are, for example, methods called support vector machines, and support vector regression. Learning here is also to calculate discrimination circuit weights, filter coefficients, and offsets, and besides this, is also a method that uses logistic regression processing. In a case where something is determined in a machine, it is necessary for a human being to teach how determination is made to the machine. With this embodiment, determination of an image adopts a method of performing calculation using machine learning, and besides this may also use a rule-based method that accommodates rules that a human being has experimentally and heuristically acquired.

Next, operation of the cell observation device will be described using the flowchart shown in FIG. 5. This flowchart is realized by a CPU that has been provided in the control section 21 within the cell observation device 1 controlling each section within the cell observation device 1 in accordance with a program that has been stored in memory.

If the flowchart for the cell observation device shown in FIG. 5 is commenced, first of all a communication standby state is entered (S1). Here, the control section 21 awaits commencement of communication from the information terminal 100. Specifically, in the event that the user provides instruction to the cell observation device 1 that has been arranged inside a chamber that is isolated from the outside, such as an incubator, the information terminal 100 is operated. This step is a state of awaiting receipt of a control signal based on this operation, using wireless communication.

Next, it is determined whether or not power supply on/off communication has been performed (S3). Here, the control section 21 determines whether or not a communication section and a determination function have been activated at a specified time interval (for example, an interval of one minute), and whether or not there is communication from the information terminal 100. As was described previously, with this embodiment power supply for the cell observation device 1 is supplied using a battery, and so in order to prevent consumption of the power supply battery it is possible for the user to perform a power supply on or power supply off instruction from the information terminal 100 (refer to S39 in FIG. 6A). It should be noted that communication may also be performed not using normal communication, but using another energy saving communication, such as BLE (Bluetooth Low Energy).

If the result of determination in step S3 is that there has been power supply on/off communication, imaging on/off processing is performed (S5). Here, the control section 21 turns the power supply of the cell observation device 1 off if the power supply was on, and conversely turns the power supply of the cell observation device 1 on if the power supply was off. However, the minimum power supply needed to execute functions for determining instructions from the information terminal 100 is supplied. As a result of this power supply control it becomes possible to reduce wasteful energy consumption. If imaging on/off processing has been performed, processing returns to step S1.

If the result of determination in step S3 is not power supply on/off communication, it is determined whether or not various wireless communication information has been acquired (S7). If the user performs various settings by operating the operation section 115 of the information terminal 100, this setting information is transmitted by wireless communication from the communication section 114 of the information terminal 100 (refer, for example, to S45 in FIG. 6A). Also, information that is necessary to imaging is also transmitted by wireless communication from the communication section 114 (refer to S45 in FIG. 6A). For example, as information that is transmitted here there is information relating to transmission destination of the image data, conditions for at the time of shooting, various parameters, and measurement conditions for when measuring the specimen 81 etc. Also, as information that is transmitted, acquisition position information for cell images from the image input section 23a of the cell observation device 1 based on positions where colonies that occurred have been inferred by the inference engine 111b is also included. In this step it is determined whether or not these settings and information have been received by the communication section 24 within the cell observation device 1.

If the result of determination on step S7 is that various wireless communication information has been acquired, information acquisition, various setting and communication etc. are performed (S9). In this step the control section 21 performs various settings within the cell observation device 1 based on various information and settings that have been acquired by the communication section 24.

Once the information acquisition, various settings and communication etc., have been performed in step S9, it is next determined whether or not a manual position designation has been received (S11). There may be cases where the user designated shooting position before observing, measuring or shooting the specimen 81 within the cell vessel, or while observing, measuring or shooting the specimen 81, or wants to observe an image at that position. In this case, the user can designate shooting position by operating the information terminal 100 (refer to S49 in FIG. 6A). In this step, the control section 21 determines whether or not wireless communication for performing this manual position designation has been received. It should be noted that positions of colonization may also be received (refer to S63 in FIG. 6B).

If the result of determination in step S11 is that manual position designation has been received, imaging is performed at the designated position, and imaging results are transmitted (S13). Here, control signals are output such that the movement section 22 will move the camera section (imaging unit) 10 to the manual position that has been received by wireless communication. The movement section 22 performs drive control of the Y actuator 31a and the X actuator 31b to move the camera section 10 to the manual position that has been designated. As a position that has been designated there should be an initial position, or a location with no risk to the camera section 10, such colliding with an obstacle etc.

If images that are a result of imaging have been transmitted in step S13, or if the result of determination in step S11 was that manual position designation was not received, it is next determined whether or not a measurement commencement signal has been received (S15). If the user commences measurement such as counting a number of cells of the specimen 81 within the cell vessel 80, and whether or not a colony is being formed, etc., that fact is instructed to the cell observation device 1 (refer to S53 in FIG. 6B). Here, the control section 21 determines whether or not a measurement commencement signal to instruct commencement of this measurement has been received. If the result of this determination is that a measurement commencement signal has not been received, processing advances to step S21.

If the result of determination in step S15 is that the measurement commencement signal has been received, images at positions corresponding to a scan pattern are acquired, and the images that have been acquired are transmitted (S17). Here, the control section 21 moves the camera section 10 in accordance with a movement pattern 25a that is stored in the storage section 25, and the image input section 23a acquires cell images at individual shooting positions. If the cell observation device 1 has acquired cell images, those images are transmitted to the information terminal 100.

The information terminal 100 infers positions where it is predicted colonies will occur using the inference engine 111c, and colony occurrence predicted positions are transmitted to the cell observation device 1 based on the result of this inference (refer to S63 in FIG. 6B). If the cell observation device 1 receives these predicted positions (refer to S9), a scan pattern is changed in accordance with the predicted positions. For example, in order to cancel shooting at positions where it is predicted that a colony will not occur, shooting is only performed at positions where it is predicted a colony will occur. Even at positions where it is predicted that a colony will occur also, in a case where the colony is not at the center of a shooting screen, adjustment of shooting position is performed so that the colony is at the center.

Adjustment of shooting position will be described using FIG. 11A and FIG. 11B. FIG. 11A shows a cell image observed using the imaging section (photographing lens and image sensor of the image input section 23a). Coordinates of the shooting center position of the imaging section are X=X1, Y=Y1, and an imaging range is Xa, Ya. A case is shown where colony C is predicted by the inference engine 111c. Colony C is elliptical, and length in the major axis direction is made (½)Ya, while length in the minor axis direction is made (½)Xa. As a result, the center position of the colony C is offset by ΔX=(¼)Xa, ΔY=(¼)Ya from the shooting center position X1, Y1. When acquiring cell images using the image input section 23a, it is desirable to form an image of a location where the colony will occur. Therefore, the camera section 10 (imaging section) is moved in accordance with a scan pattern, and the position of a shooting center at the time of shooting cells is adjusted by ΔX, ΔY, as shown in FIG. 11B.

Also, if the control section 21 has received designation of time lapse from the information terminal 100 in step S17, imaging control of a colony is performed at specified time intervals by the imaging section (refer to S63 in FIG. 6B, and to FIG. 9C). For the purpose of time lapse display, the control section 21 takes pictures of colonies at colony positions that have been predicted, at specified time intervals.

Next, it is determined whether or not imaging and measurement are complete (S19). Here, the control section 21 determines whether or not imaging and measurement have been completed in accordance with all movement patterns 25a that are stored in the storage section 25 (also including cases where there has been change in accordance with predicted position). If the result of this determination is that imaging and measurement have been completed, processing returns to step S7 and the previous operations are executed. In the event that the user operates the operation section 115 during measurement, and various settings, designation of manual position, or an image request has been performed, processing is executed in accordance with these instructions.

If the result of determination in step S19 is completion, or if the result of determination in step S15 is that a measurement commencement signal is not received, it is determined whether or not an image request will be issued (S21). There are cases where, after completion of measurement or before commencement of measurement, the user wants to browse images that have been acquired in the cell observation device 1. In this case the operation section 115 of the information terminal 100 is operated to request images. In this step the control section 21 determines whether or not there has been this image request. Also, in the above described loop, images that have been reduced in accordance with the live view display of the digital camera, taking into consideration speed of image processing and communication, are transmitted, and priority may be given to confirming promptly. On the other hand, in this step the control section 21 may also temporarily stores images of higher resolution, such as stored images in the digital camera and then transmit such images of a comparatively large size. Specifically, the imaging section performs fine scanning at specified locations, the control section 21 temporarily stores results of having performed so-called super resolution processing in the storage section 25, and high-resolution images may be transmitted and output externally at this time.

If the result of determination in step S21 is that there has been an image request, stored images are subjected to wireless transmission (S23). Here, the control section 21 transmits cell images that were acquired in step S11 and stored in the storage section 25 to the information terminal 100. If the control section 21 has transmitted stored images, or if the result of determination in step S21 was that there was not an image request, processing returns to step S1 and the previously described operations are executed.

In this way, in the flow for the cell observation device the cell observation device 1 transmits images of cells that have been imaged to the information terminal 100 (refer to S17). If a position where a colony is being generated or where it is predicted that a colony will be generated is received from the information terminal 100, a scan pattern is changed in accordance with this position (refer to S17).

Next, operation of the information terminal will be described using the flowcharts shown in FIG. 6A and FIG. 6B. This flowchart is executed by the CPU provided in the control section 111 within the information terminal 100 controlling each section within the information terminal 100 in accordance with programs stored in memory.

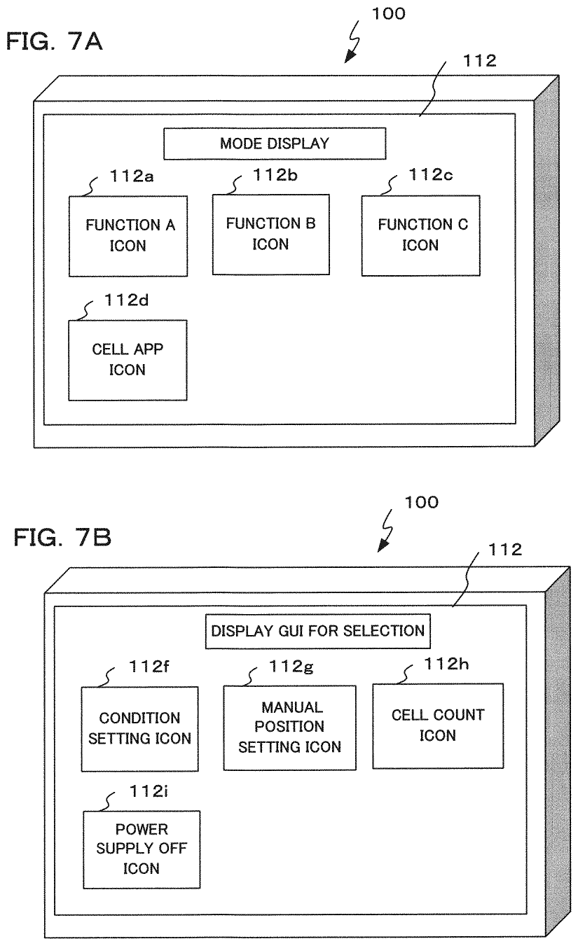

If the flow for information terminal communication is entered, first, mode display is performed (S31). Here, the control section 111 displays modes that are capable of being set in the information terminal 100 on the display section 112. For example, as shown in FIG. 7A, a function A icon 112a, function B icon 112b, function C icon 112c, and cell app icon 112d, that can all be set in the information terminal 100, are displayed, as shown in FIG. 7A. The cell app is an application relating to this embodiment, and is software that is suitable for cell cultivation, such as condition setting at the time of counting a number of cells or taking pictures of cells, inference of cell colonies etc. Modes such as function A are phone function, mail function etc. if the information terminal 100 is a smartphone. Also, if the information terminal 100 is a personal computer that is used by a scientist, there are an image analysis function, a scientific article writing support function etc.

If mode display has been performed, it is next determined whether or not the cell app will be launched (S33). If modes have been displayed, the user can select either of the modes by performing a touch operation etc. from within that mode display. In this step, the control section 111 determines whether or not the cell app has been selected and launched, from among the plurality of modes that are displayed. If the result of this determination is that the cell app will not be launched, the mode (function) that has been selected is executed. If a mode has not been selected, a standby state in entered in a state where mode display has been performed. It should be noted that selection of the modes is not limited to a touch operation, and the modes may also be selected by operating an operation member of the operation section 115.

If the result of determination in step S33 is launch of the cell app, a GUI for selection is displayed (S35). Here, the control section 111 displays selection items for the case where the cell app will be used on the display section 112. For example, a condition setting icon 112f, manual position setting icon 113g, cell count icon 112h, and power supply off icon 112i are displayed on the display section 112, as shown in FIG. 7B. The condition setting icon 112f is an icon for setting shooting conditions etc. The manual position setting icon 112g is an icon for observing and shooting cells, at a position that has been designated by the user. The cell count icon 112h is an icon for causing the number of cells to be automatic counted. The power supply off icon is an icon for turning power supply of the cell observation device 1 off. The user can select any from among these icons by means of a touch operation etc.

If the GUI for selection has been displayed, it is next determined whether or not there is imaging off (S37). Here, the control section 111 determines whether or not the user has selected the power supply off icon 112i from within the GUI display (refer to S35 and FIG. 7B).

If the result of determination in step S37 is that a power supply off operation has been performed, an on/off signal is transmitted (S39). Here, the control section 111 transmits the power supply on/off signal to the cell observation device 1 by means of the communication section 114 (refer to S3 and S5 in FIG. 5). Once the on/off signal has been transmitted processing returns to step S35.

If the result of determination in step S37 is not power supply off, it is next determined whether or not there is condition setting (S41). Here, the control section 111 determines whether or not the user has selected the condition setting icon 112f from within the GUI display (refer to S35 and FIG. 7B).

If the result of determination in step S41 is conditions setting, setting conditions are determined (S43). If the user has selected the condition setting icon 112f from within the GUI display, the control section 111 displays a plurality of icons representing various setting conditions on the display section 112. As various setting conditions there are, for example, image transmission destination, shooting conditions, shooting parameters, and measurement conditions. Since the user selects conditions they want to set from among these items, in this step the control section 111 determines which icons have been selected.

If the result of determination in step S43 is setting, various settings are performed (S45). Here, the control section 111 displays setting conditions that have been selected. For example, in a case where image transmission destination has been selected as a setting condition, the information terminal 100 or PC or server etc. that are external to the information terminal 100 are displayed on the display section 112 as transmission destinations for images that have been acquired by the cell observation device 1, and the user selects from among these transmission destinations. Also, in a case where shooting conditions has been set as a setting condition, exposure control values and focus adjustment (automatic or manual) etc. are displayed, and the user selects or sets numerical values from among these displayed options.

If various settings have been performed in step S45, or if the result of determination in step S43 was not settings, or if the result of determination in step S41 was not condition settings, it is determined whether or not there is a manual operation input (S47). Here, the control section 111 determines whether or not the user has selected the manual position setting icon 112g from within the GUI display (refer to S35 and FIG. 7B).

If the result of determination in step S47 is manual operation input, imaging is instructed at the designated position, and acquisition results are displayed (S49). Here, the control section 111 displays an input screen for the user to designate imaging position, on the display section 112. As an input screen imaging position may also be designated using x, y coordinates. If an imaging position has been designated, the control section 111 transmits the designated position to the cell observation device 1 by means of the communication section 114. Once the imaging position has been received the cell observation device 1 performs imaging at that position, and transmits images that have been acquired to the information terminal 100 (refer to S13 in FIG. 5). Once acquired images have been received, the information terminal 100 displays the acquired images on the display section 112.

In a case where acquired images have been displayed in step S49, or if the result of determination in step S47 is that there was not manual operation input, it is next determined whether or not there is cell count (S51). Here, the control section 111 determines whether or not the user has selected the cell count icon 112h from within the GUI display (refer to S35 and FIG. 7B).

If the result of determination in step S51 is cell count, a commencement signal is transmitted to the cell observation device 1 (S53). Here, the control section 111 transmits a commencement signal for commencement of measurement of number of cells to the cell observation device 1 by means of the communication section 114. If the cell observation device 1 receives the commencement signal (S15 in FIG. 5), the image input section 23a acquires images of cells, and transmits the images that have been acquired to the information terminal 100 (refer to S17 in FIG. 5).

If the commencement signal has been received in step S53, or if the result of determination in step S51 is not cell number count, it is determined whether or not measurement results have been received (S55). As was described previously, if images of cells have been received by the image input section 23a, the cell observation device 1 transmits these images to the information terminal 100 (refer to S17 in FIG. 5). In this step the control section 111 determines whether or not images have been received from the information terminal 100.

If the result of determination in step S55 is that measurement results have been received, a number of cells is counted and prediction results are displayed (S57). Here, the control section 111 counts a number of cells based on the images that were acquired by the cell observation device 1, and predicts change in the number of cells from now on. Prediction of the future number of cells may be sought using the inference engine 111c, and may be sought using linear prediction calculations from previous results.

The control section 111 displays the number of cells that has been counted, and the predicted future change, on the display section 112 in step S57. For example, with the example shown in FIG. 2, graphs 112a showing change in number of cells over time, for each location within the cell vessel 80, are displayed on the display section 112. In the graph 112a, a solid line represents actual change in number of cells, and the dashed line represents expected change in number of cells in the future. With the example shown in FIG. 2, there are only three positions within the cell vessel 80, but there may be four or more, or one, or two. The positions that are displayed may be changed cyclically every time a touch operation is performed on an icon, not shown.

Next, it is determined whether or not to return to the previous screen (S59). There will be cases where the user will want to return to the previous screen, after cell number count results and future predictions have been displayed in step S57. In this case, the user performs a touch operation on the return icon 112c (refer to FIG. 2) on the display section 112. If the result of determination in this step is that a return operation has been performed, step S55 is returned to, and the previous screen is displayed.

On the other hand, if the result of determination in step S59 is not to return, it is determined whether or not there is colony inference result confirmation (S61). As was described using FIG. 1A to FIG. 1E, cells aggregate and form colonies, but it is not easy to predict locations where colonies will occur. With this embodiment, therefore, locations where colonies will occur are inferred by the inference engine 111c using an inference model. In this step it is determined whether or not it has been possible to infer locations where colonies will occur. If the result of this determination is that it is not possible to infer locations where colonies will occur, processing advances to step S67.

If the result of determination in step S61 is that it was possible to infer locations where colonies will occur, transmitting of position information, displaying of results on monitor, displaying of plural comparisons, and displaying of size determination combination, are performed (S63). Here, the control section 111 first transmits colony occurrence predicted position information to the cell observation device 1. If the cell observation device 1 acquires predicted position information for colony occurrence during measurement (S11 in FIG. 5), a scan pattern is changed so as to perform shooting at the positions that have been predicted (S17). In this way it is possible to skip imaging at positions where colonies do not occur, and it is possible to perform measurement with good efficiency. However, taking into consideration prediction accuracy skipping may not be performed immediately.

The control section 111 also displays positions where colonies occur on the display section 112. For example, with the example shown in FIG. 2, an icon 112d indication “colonization” is displayed at a location where it is predicted that a colony will occur. In FIG. 2, since it is predicted for location 3 that cells will become a colony, a colonization icon 112d is displayed at a position corresponding to location 3. Since it is not predicted that a colony will occur for location 1 and location 2, the colonization icon 112d is not displayed as positions corresponding to these locations.

Also, the control section 111 may also perform display of cell images for locations that will be colonized. For example, if the colonization icon 112d shown in FIG. 8A is subjected to a touch operation, the display section 112 displays cell image 112j, as shown in FIG. 8B. The user can observe the state of cells at the current time by displaying the cell image 112j. Also, as a result of a touch operation of the predictive display icon 112m, a cell image for the future is displayed after a specified time. This predictive image is inferred using the inference engine 111c, and this inference result should be displayed. Also, what will happen to the cell count result after this (for example, change of increase or decrease in the number of cells in a colony etc.) may be displayed, and predictive display of good or bad (for example, will a colony change in a good direction or change in a bad direction in the future etc.) may also be performed.

Also, a time lapse icon 112k is displayed on the display section 112. If the user operates the time lapse icon 112k cell images are stored at specified time intervals for a designated location (with the example of FIG. 8B, location 3). By sequentially displaying stored images, it is possible to observe a time lapse image. Also, when the time lapse icon 112k has been operated, the control section 111 reads out images that have been stored in the recording section 25 and may perform time lapse display based on the stored images up to the current time.

The control section 111 may also perform multiple comparisons. The multiple comparison here is comparison and display of cell images that have been formed at a plurality of locations. For example, with the example shown in FIG. 9A, in a case where it is inferred that a colony will occur at location 2 and location 3, colonization icons 112d2 and 112d3 are displayed for respective locations. In this case, if the user operates the colonization icons 112d2 and 112d3 cell images 112j2 and 112j3 corresponding to respective locations will be displayed, as shown in FIG. 9B.

Further, in the state shown in FIG. 9B, if the user operates the time lapse icon 112k, time lapse display will be performed for 6 hour intervals (refer to interval display 112n), as shown in FIG. 9C. Most recent cell images 112j2a and 112j3a are display to the front of the screen, and cell images 112j2b, 112j23b for six hours previous are displayed behind respective images. In a state where this comparison display has been performed, the user may switch an image that is displayed at the back to the front of the screen by operating the time lapse icon 112k again, for example. When shooting time lapse images, it is desirable for the cell observation device 1 to shoot images under the same conditions, in order to make comparison of change possible.

Also, the control section 111 determines size, and may also perform composite display based on results of this determination. This composite display is combining two cell images shown in FIG. 10B, and displaying a single combined image as shown in FIG. 10C. There are cases where, depending on a relationship between a position of the imaging section of the image input section 23a (photographing lens and image sensor) and a position of a cell colony on obtaining cell images, a single colony appears in two separate images. With the example shown in FIG. 10B, a single colony is depicted in images F6 and F7. In this case, an outline of the colony is extracted, images F6 and F7 are combined so that outlines of images F6 and F7 are connected, and a combined image F8 is generated such as shown in FIG. 10C. This composite display may be automatically determined using features of a plurality of images, and in a case where there is protrusion from an appropriate position that has been predicted from conditions before growth with cultivation, multiple shooting combination may be performed from image acquisition the next and subsequent time. This type of prediction being possible can also be said to be a technical effect of this embodiment. Also, composite mode may be set not automatically but manually, and such manual setting of composite mode may be performed at this time by providing a switch so that the user can perform designation of shooting position and number of combinations.

Combined image F8 resulting from having combined a plurality of images is displayed on the display section 112, as shown in FIG. 10A. At the time of display of combined image F8, the control section 111 may be made to display coordinates (monitor coordinates) of the imaging section corresponding to the combined image on the display section 112.

Also, if the inference engine 111c of the control section 111 infers position where a colony will occur, this position is transmitted to the cell observation device 1. As was described earlier, when the imaging section is moved in accordance with a specified scan pattern, the cell observation device 1 corrects imaging position based on this position where a colony is predicted to occur (refer to S17 in FIG. 5).

If the processing of step S63 has been executed, it is next determined whether or not to return to the previous screen (S65). There will be cases where, after having executed any of the processes accompanying colony inference in step S63, the user will want to return to the previous screen. In this case, the user performs a touch operation on the return icon 112c on the display section 112. If the result of determination in this step is that a return operation has been performed, step S61 is returned to, and the previous screen is displayed.

On the other hand, if the result of determination in step S65 is that a return operation has not been performed, or if the result of determination in step S61 is that a result of colony inference is that a colony could not be confirmed, or if the result of determination in step S55 is that measurement results were not received, it is determined whether or not the app is to be terminated (S67). Here, the control section 111 determines whether or not an instruction to terminate operation of the cell app, that was launched in step S33, has been issued. If the result of this determination is not to terminate the cell app, processing returns to step S35, while if the result of determination is to terminate the cell app processing returns to step S31.

In this way, with the flow for information terminal communication, positions where it is predicted that a colony will occur are inferred based on cell images that have been transmitted from the cell observation device 1 (refer to S61). Then, in a case where it has been predicted that a colony will occur, that position is transmitted to the cell observation device 1 (S63), and it is made possible to change a scan pattern in the cell observation device 1 (S17). Also, inference results are displayed on the display section 112 (refer to S63, and the graphs for locations 1 to 3 in FIG. 2 and FIG. 8 etc.). It is also made possible to compare and display images of a plurality of locations (refer to FIG. 9B). Time lapse display is also made possible (refer to S63 and to FIG. 9C). Also, in a case where colonies span across a plurality of images, parts of the colonies are combined and displayed (refer to S63 and to FIG. 10B).

Next a modified example of colonization determination will be described using the flowchart shown in FIG. 12. With the one embodiment of the present invention, prediction of positions where cell colonies will occur was performed by the inference engine 111c. Specifically, deep learning was performed using training data relating to occurrence positions of colonies, an inference model was generated, and the inference engine 111c predicted positions where colonies would occur using this inference model. However, besides inference using the inference engine 111c, colony occurrence positions may also be predicted by the CPU within the control section 111 in accordance with a program. The flowchart shown in FIG. 12 predicts colony occurrence positions using a program.

If the flow for colonization determination shown in FIG. 12 is commenced, first, inter cell determination is performed (S71). Here, the control section 111 acquires stopped position and angle of view information of the imaging section. Specifically, the control section 111 acquires stopped position of the imaging section (camera section 10) that has been acquired by the position input section 23b from the cell observation device 1 and angle of view information of the photographing lens from the angle of view information 25b in the storage section 25. Next, the control section 111 analyzes cell images from the cell observation device 1, and extracts outlines of cells to determine positional relationships between cells.

Next, it is determined whether or not there is no change in position relationship between cells over a specified time (S73). Here, the control section 111 determine a positional relationship between cells every time a cell image is input from the cell observation device 1. The control section 111 determines whether or not there is no change in this positional relationship over a specified time. If the result of this determination is that there is no change, a colony has not formed, and so step S71 is returned to.

On the other hand, if the result of determination in step S73 is that there is change in a positional relationship between cells, it is determined whether or not the change is cell division or cell binding (S75). As was described using FIG. 1A to FIG. 1E, when a colony of cells is formed, there is cell division, and there is combination of a plurality of cells. Here, the control section 111 performs determination by analyzing cell images. If the result of this determination is that there is no cell division or cell binding then a colony is not being formed, and so step S71 is returned to.

On the other hand, if the result of determination in step S75 is that there is cell division or cell binding, it is determined that there is colonization (S77). Since there is change in positional relationship between cells, and also there is cell division or cell binding, the possibility of a colony forming is high. The control section 111 therefore determines that cells are colonizing. Information on position where a colony is being formed is associated with this colonization determination information. If colonization has been determined, this flow is terminated.

In this way, with colonization determination an inference engine may be used, and it is also possible for a CPU to predict positions where colonies will occur in accordance with a program. Specifically, colony formation is predicted by performing image analysis of cell images and analyzing change over time in cell images and positional relationships between cells. It should be noted that the flow shown in FIG. 12 performed colonization prediction within the information terminal 100. However, this is not limiting and it is also possible to perform colonization determination in the control section 21 within the cell observation device 1. Here, whether or not cell cultivation has processed as expected is determined based on colony formation. However, the fact that cell cultivation is proceeding as expected may also be determined with a method other than this method. In other words, if there are time series imaging results and cell count data for the same culture positions as where a culture that was cultivated as expected was obtained, then since training data for creating an inference model can be obtained from characteristics of that change, prediction determination for cultivation as expected may also be performed using this inference model.

Also, if there is a purpose where efficiency of ascertaining whether a culture is good or bad ahead of time has been assumed, a case where progress is not as expected may be determined, and in this case also, a similar approach can be applied. That is, if there is time series imaging results and cell count data for the same culture positions as where a culture that was not cultivated as expected was obtained, training data for creating an inference model for inferring that cultivation will not be performed well is obtained from characteristics of that change, and so it is also possible to perform prediction determination for cultivation not proceeding as planned using this inference model. In cultivation of cells there are many cases where disruptions arise such as problems with temperature and humidity management, disturbance of light etc., and contamination etc., and there are many cases where ascertaining these situations in advance at an early stage is difficult even for a specialist.

As has been described above, with the one embodiment and modified example of the present invention, a cell observation system has an imaging section that is capable of movement in the horizontal direction, and that form images of cells that have been cultivated in a vessel. Then, position of the imaging section is changed by moving in the horizontal direction (refer to S17 in FIG. 5), colonies are determined based on images of cells that have been acquired by the imaging section (refer to S61 in FIG. 6B), and position of a colony is determined based on movement position of the imaging section and imaging range, at the time a colony was determined (refer to S63 in FIG. 6B). This means that it becomes possible to change imaging location in accordance with change in cultivation state (colony generation state) of cells, when imaging cells etc. within the cultivation vessel. Here, horizontal direction may also be expressed as a direction that is orthogonal to the optical axis direction of an imaging lens of the camera, or as a direction that is displaced from that direction.

Also, with the modified example and the one embodiment the present invention, the cell observation system comprises an imaging section that is capable of moving in a horizontal direction (refer, for example, to the image input section 23a in FIG. 3), and a processor (refer for example to the control section 111 in FIG. 3) that infers positions where there is generation of growth of colonies from position information or shape information of a plurality of cells within images, based on images that have been acquired by the imaging section (refer, for example, to the inference engine 111c in FIG. 3, S61 in FIG. 6B, and to FIG. 12), and controls position of the imaging section so as to form images at the inferred positions (refer, for example, to S63 in FIG. 6B). Specifically, the cell observation system of this embodiment can infer positions where colonies will be generated in a state where a colony is not formed, and infer positions where colonies will grow in a state where colonies have formed. The cell observation system therefore performs control so as to move the imaging section to the positions that have been inferred. As a result, it is possible to image colonies with good efficiency.

It should be noted that with the modified example and one embodiment of the present invention, colonization has been determined in the information terminal 100 based on images that have been acquired in the cell observation device 1. However, this is not limiting and it is also possible to determine colonization within the cell observation device 1, and change a scan pattern based on result of this determination. The information terminal 100 and the cell observation device 1 may be integratedly formed.

Also, with the modified example and one embodiment of the present invention, the whole of the imaging unit was described as a type that moves. However, this is not limiting and as a method of moving observational field of view and shooting field of view (changing position of observation and shooting) there is also a method realized by making only an image sensor and a lens moveable. Obviously, the same results can also be achieved by moving a stage etc., on which the specimen is mounted. Also, it is not always necessary to have scanning accompanying mechanical movement, and there is also a method where wide angle imaging is performed, and super resolution processing is performed only for specific parts within a field of view. The modified example and one embodiment of the present invention, can also be applied to such a system or method, or to a unit or device that uses such a system or method. Accordingly, there may also be provided an imaging section that forms images of cells cultivated in a vessel, and a determination section that determines, or predicatively determines, colonies based on images of cells that have been acquired by the imaging section by changing imaging position as a result of moving imaging position in an optical axis direction of an imaging lens constituting the imaging sections.

Also, with the modified example and one embodiment of the present invention, the control section 21, movement section 22, information acquisition section 23, communication section 24, and storage section 25 within the cell observation device 1 are constructed separately, but some or all of these sections may be configured as software, and may be executed using one or a plurality of CPUs and their peripheral circuits. Also, with the modified example and one embodiment of the present invention, the control section 111, display section 112, information acquisition section 113, and communication section 114 within the information terminal 100 are constructed separately, but some or all of these sections may be configured as software, and may be executed using one or a plurality of CPUs and their peripheral circuits. It is also possible for each of the sections within the cell observation device 1 and the information terminal 100 to have a hardware structure such as gate circuits that have been generated based on a programming language that is described using Verilog, and also to use a hardware structure that utilizes software such as a DSP (digital signal processor). Suitable combinations of these approaches may also be used.

Also, the configuration is not limited to CPUs, and may be elements that provide functions as a controller, and processes of each of the above described sections may be performed by at least one processor that has been constructed as hardware. For example, each section may also be configured with a processor that is constructed as respective electronic circuits, and may also be each circuit section of a processor that has been constructed as an integrated circuit such as an FPGA (Field Programmable Gate Array). Alternatively, functions of each section may be executed by a processor that is constructed of at least one CPU reading out and executing computer programs that have been stored in a storage medium. SD cards, USB memory, Flash memory, CDs, and DVDs may also be included in storage medium. Also, in this embodiment etc., a processor is arranged in the control section 21 within the cell observation device 1, and a processor is arranged in the control section 111 within the information terminal 100. The number of these processors may be one in each device, or may be divided into two or more. Further, there need only be a single processor if there is high speed communication between the two devices.

Also, in recent years, it has become common to use artificial intelligence, such as being able to determine various evaluation criteria in one go, and it goes without saying that there may be improvements such as unifying each branch etc. of the flowcharts shown in this specification, and this is within the scope of the present invention. Regarding this type of control, as long as it is possible for the user to input whether or not something is good or bad, it is possible to customize the embodiments shown in this application in away that is suitable to the user by learning the user's preferences.

Also, image data used within the embodiment and data relating to annotation may be managed by a terminal, and may also be managed by a storage section within a specified server on the Internet. Various data that is managed via the Internet, or some of that data, may be managed with the centralized database, and this data may also be managed in a mutual monitoring way using a decentralized (distributed) database such as a blockchain. With a centralized database, at the time some kind of problem arises, it becomes no longer possible to manage that data until fault repair of the system, but with a distributed database it is possible to reduce faults.

With a blockchain, if there is change in data that is managed, content of that processing etc. is encrypted in block units, and by distributing to each database it is made possible to share that information with everyone (blockchain). Numerical characters for network identification, block size, header information etc. are collected together in this block. With a blockchain, when newly generating a block (that is, a collection of information that will be managed with a database), design is performed so that data of the block that was generated one before is partially included, and an entire processing history is connected in a single chain, which is why it is called a chain.

Since there is the management method as described above, management such as cell cultivation where conditions change along a time axis, and a blockchain, should have consistency. For example, every time a new cultivation process image is obtained, features of that image or count results of cells are made into a block, and linked by being associated with a previous result. Specifically, image data of appearance of cell cultivation that has been formed in time series is acquired, and history of information acquired from the image data that has been acquired is managed by block generation processing for a blockchain for every acquisition time of image data. For example, an inference model used in the inference engine 111c may be generated by managing history, of information obtained from image data that was formed in time series of appearance of cell cultivation, with block generation processing of a blockchain every time the image data is acquired.

By adopting this type of inference model generating method, it is possible to manage accuracy of cell cultivation over time, for which safety is important, and it becomes possible to guarantee quality such as of colonies that have been formed and cell sheets with the process history. If there is any kind of problem it becomes possible to manage history by tracing blocks that have been linked by a chain. Also, in a case where inference is performed using images acquired in this way, and acquired data from the images (image feature amount, cell count number), if that data is not accurate over time correct inference will not be possible. Accordingly, performing inference with data that has been certified with a blockchain constitutes extremely intelligent highly precise inference technology. That is, since image data of appearance of cell cultivation that has been formed in times series is acquired, and history of information obtained from the image data that has been acquired is managed using processing to generate a block of for blockchain for every acquisition time of image data, it becomes possible to generate an inference model for which it is easy to obtain reliability in result display, even from the viewpoint of mutual observation and historical inquiry.

In other words, in order to have connections and relationships between blocks, part of a header of a prior block is encrypted and combined in the header of the new block. In the header of this new block, the header of a prior block is combined with arbitrary data such as a “hash value” that has been encrypted using a hash function, “process storage”, and after that, a “nonce”. A hash value is for summarizing data, and it is difficult to falsify because it changes significantly with data change. Also, if restriction using special rules is provided in this has value, it is necessary to determine additional data and a “nonce” (number used once: abbreviation for a numerical character that is used only one time) in order to make the hash value satisfy this restriction.

An operation to find a nonce is called mining, and an operator looking for a nonce is called a miner, and if miners that are searching for a correct nonce can connect blocks and receive rewards, administrations that are combinations of economic incentives, such as cryptocurrency, become possible. By using this “nonce” and hash together, it is possible to further increase reliability of currencies.

In order to store transactions in a decentralized way, it is necessary to provide an incentive to participants who operate (ensuring data identity with other nodes that are distributively retained) distributed computers (nodes), and so cryptocurrency is used, but it is not necessary to assume cryptocurrency if other incentives can be offered, or if the mechanism for data identity guarantee can be simplified. For example, there may be mutual observation software for blockchain in a plurality of personal computers.

Also, among the technology that has been described in this specification, with respect to control that has been described mainly using flowcharts, there are many instances where setting is possible using programs, and such programs may be held in a storage medium or storage section. The manner of storing the programs in the storage medium or storage section may be to store at the time of manufacture, or by using a distributed storage medium, or they be downloaded via the Internet.

Also, with the one embodiment of the present invention, operation of this embodiment was described using flowcharts, but procedures and order may be changed, some steps may be omitted, steps may be added, and further the specific processing content within each step may be altered. It is also possible to suitably combine structural elements from different embodiments.

Also, regarding the operation flow in the patent claims, the specification and the drawings, for the sake of convenience description has been given using words representing sequence, such as “first” and “next”, but at places where it is not particularly described, this does not mean that implementation must be in this order.

As understood by those having ordinary skill in the art, as used in this application, ‘section,’ ‘unit,’ ‘element,’ ‘module,’ ‘device,’ ‘member,’ ‘mechanism,’ ‘apparatus,’ ‘machine,’ or ‘system’ may be implemented as circuitry, such as integrated circuits, application specific circuits (“ASICs”), field programmable logic arrays (“FPLAs”), etc., and/or software implemented on a processor, such as a microprocessor.

The present invention is not limited to these embodiments, and structural elements may be modified in actual implementation within the scope of the gist of the embodiments. It is also possible form various inventions by suitably combining the plurality structural elements disclosed in the above described embodiments. For example, it is possible to omit some of the structural elements shown in the embodiments. It is also possible to suitably combine structural elements from different embodiments.

What is claimed is:

1. A cell observation system, comprising:
an image sensor capable of movement in a horizontal direction, and
a processor, wherein
the processor infers position where a colony will be generated or grown from position information or shape information of a plurality of cells within an image, based on the image of the cells that has been acquired by the image sensor, and controls position of the image sensor so as to perform imaging at the inferred position.

2. The cell observation system of claim 1, wherein:
the inferred position is a position where it is predicted that the colony will be generated.

3. The cell observation system of claim 1, further comprising:
an actuator that moves the image sensor in a horizontal direction, wherein
the processor controls the actuator so that the image sensor forms an image at a center position of a colony, based on the inferred position.

4. The cell observation system of claim 3, wherein:
the processor controls the image sensor so as to perform time lapse photography of a colony or cells at the inferred position, at specified time intervals.

5. The cell observation system of claim 1, further comprising:
a display that is capable of displaying images of cells or colonies that have been acquired by the image sensor.

6. The cell observation system of claim 5, wherein:
the processor analyzes a plurality of images that have been acquire by the image sensor at the inferred position, measures a number of cells within image, and outputs change over time in the number of cells to the display.

7. The cell observation system of claim 1, further comprising:
an inference engine that is provided with a colonization inference model that has been generated with cell images as training data, wherein
the inference model infers position where a colony will be generated, based on input of images that have been formed by the image sensor, and outputs as information on the inferred position.

8. The cell observation system of claim 7, wherein:
the inference model outputs determinations results as to whether culture is good or bad based on images or information on inferred position.

9. The cell observation system of claim 7, wherein:
the inference model is generated by managing history, of information obtained from image data of appearance of cell cultivation that was formed in time series, with block generation processing for a blockchain for every acquisition time of the image data.

10. The cell observation system of claim 1, further comprising:
memory that is capable of storing the inferred position that has been inferred by the processor.

11. An inference model generating method, comprising:
acquiring image data formed in time series of appearance of cell cultivation leading to generation of a colony of cells;
designating image portions where there is colonization, among the image data that has been acquired, as annotation, and making image data that has been designated with this annotation into training data; and
generating a colonization inference model using the training data, with input made cell images before colonization, and output made expected position of colonization.

12. The cell observation system of claim 1, further comprising:
inputting cell images before the colonization to a colonization inference model that has been generated by the inference model generating method of claim 11; and
executing computational processing using the colonization inference model, and inferring position where the colony will be generated.

13. A non-transitory computer-readable medium storing a processor executable code, which when executed by at least one processor, performs a cell observation method, the cell observation method comprising:
inferring position where a colony will be generated or grown from position information or shape information of a plurality of cells within an image, using images of cells that has been acquired by an image sensor that is capable of movement in a horizontal direction, and controlling position of the image sensor so as to perform imaging at the inferred position.
